(19) United States
(12) Patent Application Publication
(10) Pub. No.: US 2014/0288507 A1
(43) Pub. Date: Sep. 25, 2014

(54) Self-Locking Syringe

(71) Applicant: Bensson Samuel, Bethlehem, PA (US)

(72) Inventor: Bensson Samuel, Bethlehem, PA (US)

(21) Appl. No.: 13/896,387

(22) Filed: May 17, 2013

Related U.S. Application Data
(60) Provisional application No. 61/804,697, filed on Mar. 24, 2013.

(51) Int. Cl. A61M 5/315

(52) U.S. Cl. CPC A61M 5/31513
USPC 604/222

(57) ABSTRACT

A self-locking syringe assembly is disclosed. The syringe assembly includes a barrel having an interior surface. An elongate plunger shaft is axially moveable within the barrel. The plunger shaft includes at least two longitudinally extending splines. A stopper is attached to the distal tip of the plunger shaft which provides a fluid-tight seal with the barrel. Tabs are positioned at annularly spaced locations on the interior surface of the barrel. The tabs extend radially inwardly from the sidewall a predetermined distance and define recesses therebetween for receiving the plunger splines during axial movement of the plunger shaft within the barrel. The splines include recesses to enable rotational movement of the plunger shaft when the recesses are axially aligned with the tabs to dispose the splines against the tabs. In this manner, the plunger shaft may be locked in a predetermined longitudinal position relative to the barrel. Once in this locked position, the plunger shaft is prevented from being pulled or pushed towards the distal end of the barrel.

### CROSS-REFERENCE TO RELATED APPLICATIONS

This application claims priority to U.S. Provisional Application No. 61/804,697 filed Mar. 24, 2013.

### FIELD OF THE INVENTION

The invention relates generally to syringes employed in the medical field, particularly those used for injecting fluids or for aspirating fluids or tissue under the influence of a vacuum. More specifically, invention relates to a syringe employing a locking device.

### BACKGROUND OF THE INVENTION

Various designs of devices are now in use in the medical field for taking in vivo internal fluid samples, e.g., blood, body fluids such as pleural fluids, peritoneal fluids, etc. or tissue samples to perform tests on such fluid or tissue. For example, devices are in use for removing fluid or tissue from a patient by a vacuum or suction. In such devices, a plunger is moved in a hollow barrel to create a vacuum in the barrel for withdrawing the fluid or tissue from a patient's body into the tube or barrel. When this is done, a relatively strong vacuum is created within the barrel of the syringe which encourages a small amount of the fluid or tissue to enter the barrel. This vacuum also tends to exert a force on the syringe plunger that would otherwise pull the syringe plunger back into the barrel of the syringe unless the syringe plunger is restrained by grasping it tightly and holding it. Thus, the creation of a vacuum in the syringe tends to pull the plunger back into the syringe barrel unless some mechanism is employed to prevent it.

With some syringes, the practitioner merely holds onto the shank of the plunger so as to prevent it from being drawn into the barrel of the syringe. However, this is awkward, and sometimes necessitates the use of two hands, and can lead to fatigue, and prevents the practitioner from attending to other tasks. Therefore, many syringes employ some form of a locking device wherein the syringe plunger is held in a retracted position, to provide a long-lasting suction pressure without requiring the user to hold the plunger in its retracted position. The use of such locking devices reduces the potential for fatigue and frees up the user's hands for other matters.

Existing syringes that include locking features often require added components, such as locking collars, making their construction complicated. Moreover, existing syringes that include locking features often require that the plunger with its stopper attached must be distorted as the plunger is forced past the locking device while being assembled with the syringe barrel by insertion therein. This is an inconvenient technique, and can result in distortion of the plunger and/or stopper during assembly, and may increase the possibility for leakage during use, and even breakage of these components during assembly or use. Alternatively, such locking devices of existing syringes almost universally require extensive modifications of the barrel, and/or the plunger, thus making it impossible to perform simple, relatively inexpensive, modifications to existing syringes in order to add a locking device.

Thus, a need exists to provide a self-locking syringe having a minimal number of components which is simple to construct and reliable in operation and which withdraws fluid and tissue efficiently from a patient's body and utilizes a locking mechanism that holds the fluid or tissue after such withdrawal without the need for continued human intervention.

### SUMMARY OF THE INVENTION

A self-locking syringe assembly is disclosed. The syringe assembly includes a barrel having an interior surface. An elongate plunger shaft is axially moveable within the barrel. The plunger shaft includes longitudinally extending splines. A stopper is attached to the distal tip of the plunger shaft which provides a fluid-tight seal with the barrel. Several tabs are positioned at annularly spaced locations on the interior surface of the barrel. The tabs extend radially inwardly from the sidewall a predetermined distance and define recesses provided for receiving the plunger splines during axial movement of the plunger shaft within the barrel. The splines include recesses to enable rotational movement of the plunger shaft when the recesses are axially aligned with the tabs to dispose the splines against the tabs. In this manner, the plunger shaft may be locked in a predetermined longitudinal position relative to the barrel. Once in this locked position, the plunger shaft is prevented from being pulled or pushed towards the distal end of the barrel.

### DESCRIPTION OF THE DRAWINGS

FIG. 1 is a perspective view of a self-locking syringe assembly constituting one embodiment of the present invention and illustrating the plunger shaft in the forward position prior to an aspiration stroke;

FIG. 2 is an exploded perspective view of the self-locking syringe assembly constituting one embodiment of the present invention illustrating the plunger shaft, the stopper and the syringe barrel;

FIG. 3 is a cross-sectional view taken along line 3-3 of FIG. 1 with the plunger shaft in the forward position prior to an aspiration stroke;

FIG. 4 is a cross-sectional view of the self-locking syringe assembly constituting one embodiment of the present invention and illustrating the plunger shaft in a retracted position subsequent to an aspiration stroke;

FIG. 5 is a cross-sectional view taken along line 5-5 of FIG. 3; and,

FIG. 6 is a cross-sectional view taken along line 6-6 of FIG. 4.

### DETAILED DESCRIPTION OF THE PREFERRED EMBODIMENT

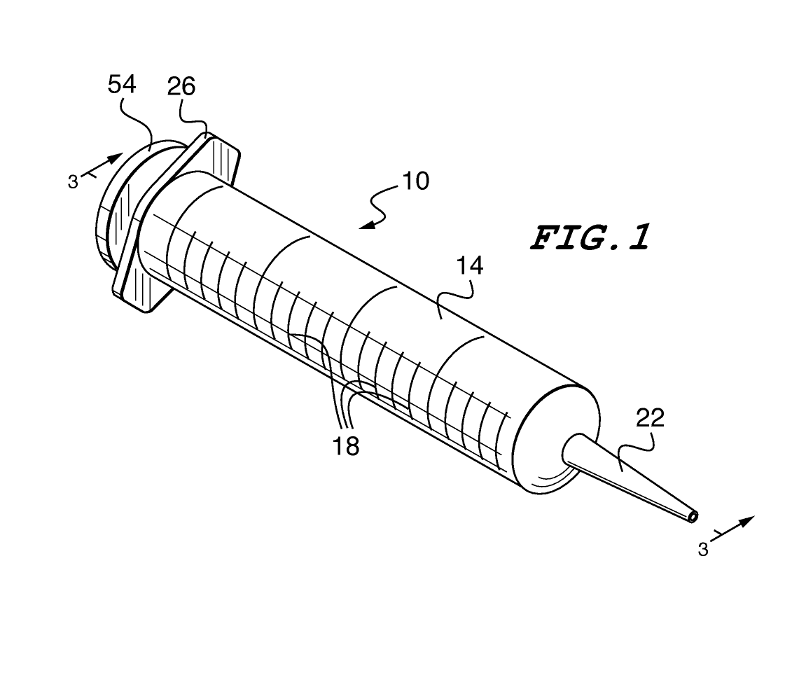

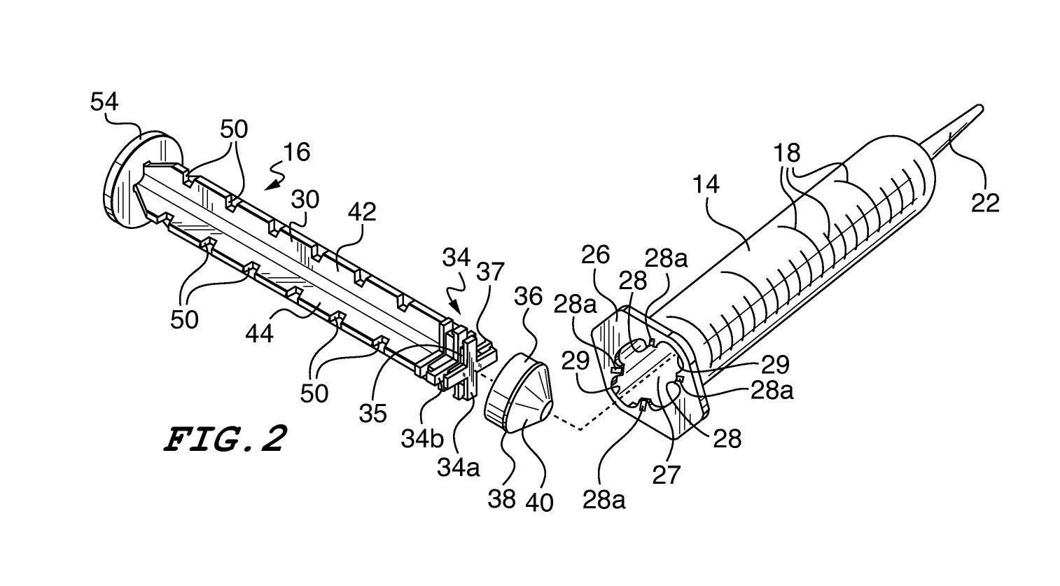

Referring now to FIGS. 1 and 2, wherein like numbers indicate like parts and components throughout the drawings, there is shown at 10 a preferred embodiment of the self-locking syringe assembly of the present invention. The syringe assembly 10 is provided for withdrawing fluid or tissue from a patient's body and for retaining the fluid or tissue in a sealed relationship after withdrawal. The assembly 10 includes a hollow barrel 14 having an open proximal end for receiving a plunger 16 therein and a distal end that is closed except for a relatively small opening designed as a standard male Luer-Lock connection for receiving a needle such as an aspirating needle 22, or a biopsy needle. The needle 22 may be provided with a cutting edge (not shown) at its distal extremity to remove tissue from a patient's body (e.g. a patient's lung or uterus).

The barrel 14 is preferably a one-piece molded plastic member, fashioned from a transparent plastic, e.g., polyethylene, and is generally cylindrical in configuration. The barrel 14 includes a predetermined internal length and diameter to form a sufficient volume for containing fluid or tissue aspirated from a patient's body such as blood or fluid from a lung. Progressive numerical indications of the amount of fluid or tissue withdrawn into the barrel 14 may be provided in a scale as indicated at 18. These numerical indications 18 enable the user determine when a prescribed amount of fluid or tissue has been aspirated from a patient.

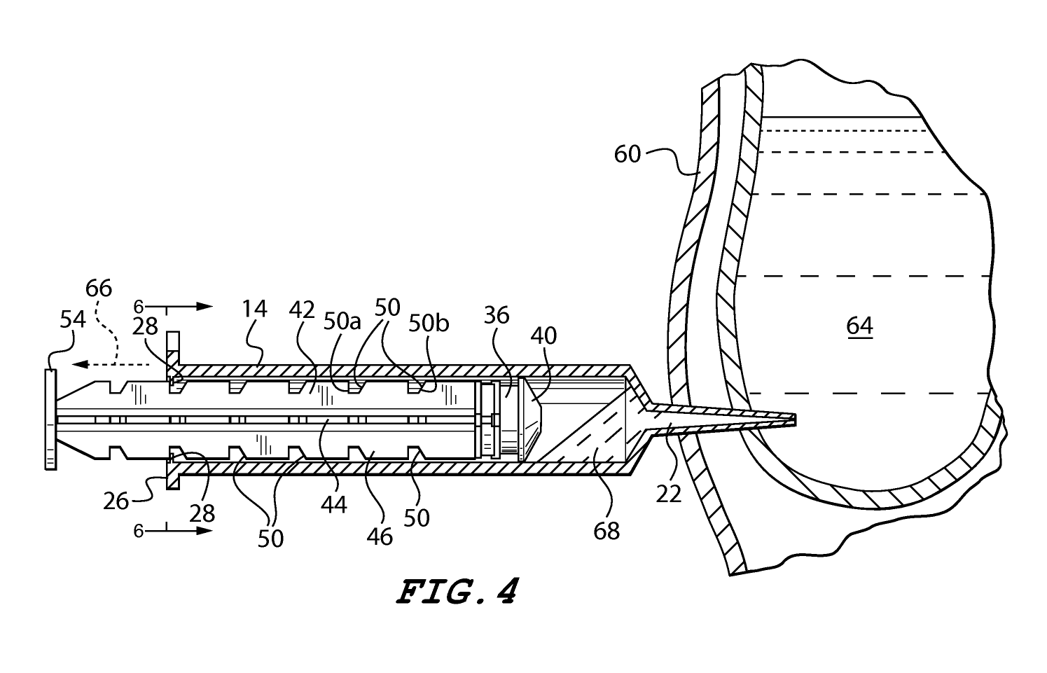

At its proximal end, the barrel 14 comprises a segmental flange 26 which extends radially outward and has a predetermined thickness. Although the flange 26 is shown as being segmental, with ears extending outwardly at positions 180° apart, the flange 26 could be completely circular if desired. The flange 26 serves as a means for supporting the fingers when withdrawing the plunger 16 from the barrel 14 as shown in FIG. 4.

Referring now to FIG. 2, at its flange 26, the barrel 14 includes a central opening 27 having a plurality, e.g., four, inwardly extending tabs 28 that are relatively rigid. The tabs 28 are positioned at locations annularly spaced apart from one another along the central opening 27. The tabs 28 define rounded slots 29 therebetween which are provided to enable passage of the plunger 16 through the proximal end of the barrel 14. As best shown in FIG. 2, four tabs 28 are shown located at equally spaced annular positions around the central opening 27 and four equally spaced rounded slots 29 are shown located between or adjacent the tabs 28. It is to be understood that the particular geometries of the tabs 28 and slots 29 are merely exemplary and variations on these geometries are within the scope of this invention.

Again referring to FIG. 2, there is shown an exploded perspective view of the plunger 16 withdrawn from the barrel 14. The plunger 16 comprises a shank 30 and includes a stopper mount 34 located at its distal end. The stopper mount 34 may include a pair of spoke structures 34a and 34b, each spoke structure formed of radially extending spokes, e.g., four radially extending spokes forming a cross-shape. The spoke structures 34a and 34b are separated by a thickness 35 located therebetween. Each spoke includes a proximally extending tip 37 to facilitate mounting of a seal or stopper 36 thereover. It should be understood that although four radially extending spokes are illustrated in the drawings, a greater or fewer number of spokes, e.g., three spokes or five spokes, could be employed without departing from the scope of the invention.

The seal or stopper 36 is arranged for mounting over the spoke structures 34a and 34b and is formed of a suitable resilient material. The spoke structures 34a and 34b provide stability and adequate support to enable the stopper 36 to sealingly engage the interior wall of the barrel 14 to create a vacuum during aspiration. In addition, during initial insertion of the plunger 16 into the open proximal end of the barrel 14, the geometry of the spoke structures 34a and 34b enable the resilient stopper 36 mounted thereover to flex or give as the stopper 36 passes across the relatively rigid inwardly extending tabs 28 located at the open proximal end of the barrel 14. In this manner, during initial assembly, the plunger 16 with the stopper 36 mounted thereon may be inserted into the barrel 14 in a manner such that the stopper 36 may pass the tabs 28 as the plunger 16 is pushed to the distal end (forward position) of the barrel 14 prior to an aspiration stroke. Under prior art syringe assemblies, a disc would customarily be provided at the distal end of the plunger for mounting the stopper thereover. Such a disc configuration may preclude the stopper from sufficiently flexing to allow passage through the inwardly extending tabs 28.

The stopper 36 may be equipped with one or more annular ridges 38. These ridges 38 have a diameter slightly greater than the internal diameter of the barrel 14, and thus, being resilient, serve to sealingly engage the interior wall of the barrel 14. Their purpose is to provide means whereby an at least partial vacuum can be established and maintained for an extended period of time in the barrel distal of the stopper 36 upon refraction of the plunger 16, as well as provide a fluid-tight seal when the contents of the barrel 14 are being expelled. End wall 40 of the stopper 36 is preferably somewhat tapered so as to form a cone-shaped outer surface.

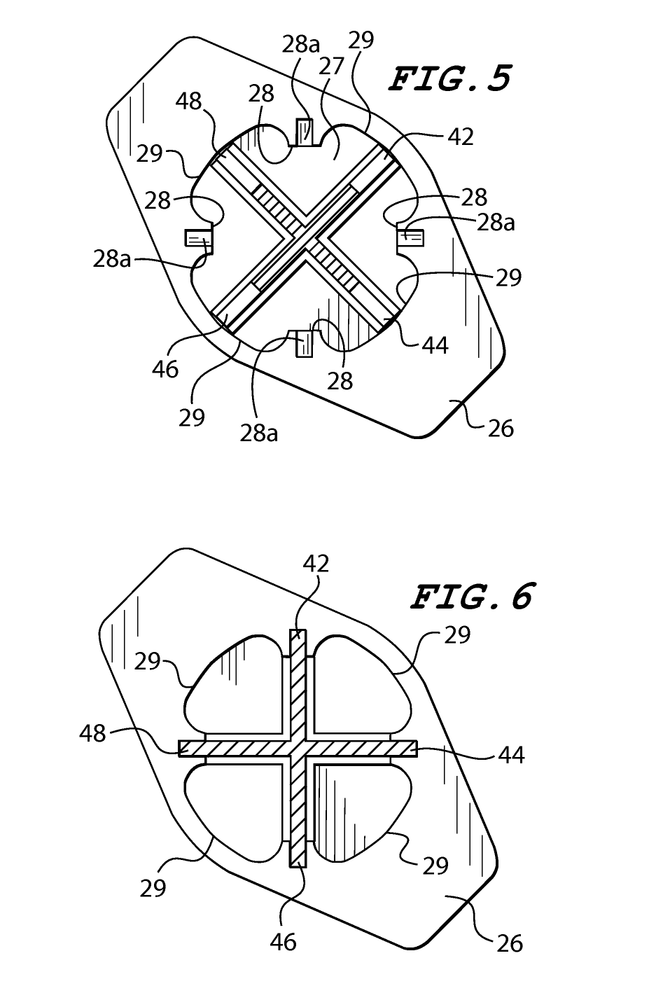

The plunger 16 is preferably fabricated as a one-piece molded item, fashioned from a rigid plastic such as polyethylene. In order to conserve material, and to reduce weight, the plunger 16 is configured with a plurality of longitudinal and radially extending splines. Preferably, there are four such splines 42, 44, 46 and 48 forming an “X” pattern as shown in cross section in FIGS. 5 and 6. Alternatively, there may be only three splines forming a “Y” pattern, or even some other number if preferred. These splines extend longitudinally over the major portion of the plunger 16 in order to provide desired rigidity. Recesses 50 are disposed in the splines 42, 44, 46 and 48 at predetermined spaced positions in the axial direction. The position of the recesses 50 along the length of the splines may correspond with the numerical indications provided on the scale 18. Each recess 50 in the spline includes a straight edge 50a and an inclined edge 50b. The widths of the recesses 50 may be slightly greater than the thickness of the flange 26.

A disc 54 is disposed on the plunger 16 at the proximal end thereof. The primary purpose of the disc 54 is to serve as a support for retracting the plunger 16 with respect to the barrel 14. Alternatively, in another mode of usage, the disc 54 may serve as a support for the thumb of a user to bear against when pushing the plunger 16 into the barrel 14, in which case the middle finger and index finger of the user bear against the distal surface of the flange 26. The proximal surface of the disc 54 is preferably serrated to aid in such usage.

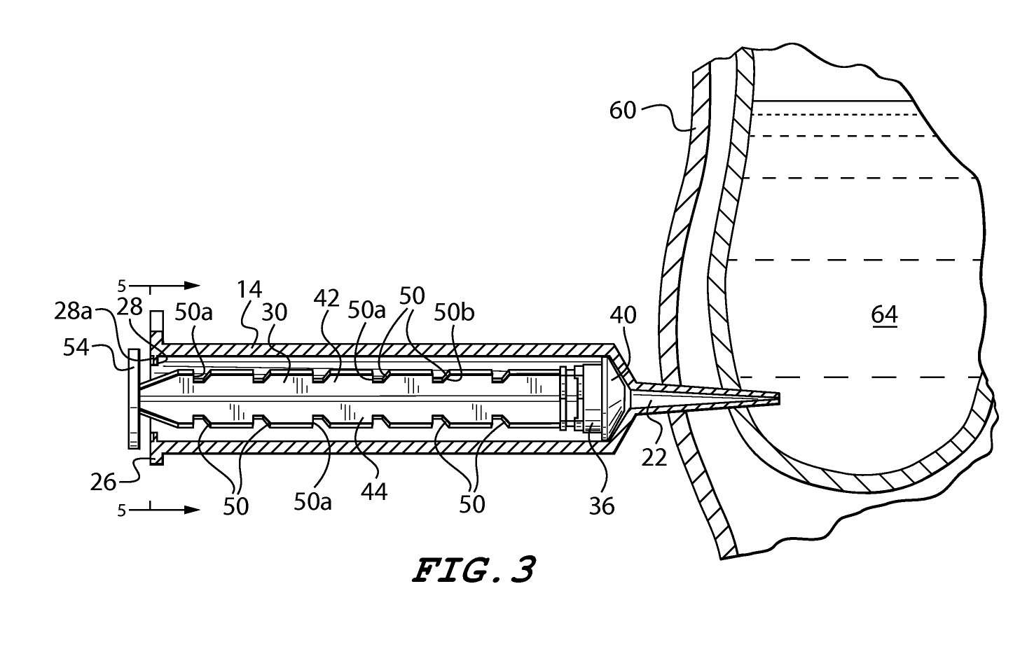

Referring now to FIGS. 3 and 4, in use, the needle 22 is disposed through the skin 60 of a patient's body and through the patient's lung, uterus or other organ 64. As shown in FIG. 4, the plunger 16 is then moved axially from the forward position in the proximal direction as indicated by arrow 66 to withdraw fluid or tissue 68 from the organ 64 or elsewhere from the patient and into the barrel 14. This creates a partial vacuum or a suction force in the barrel 14 and the needle 22 to withdraw fluid or tissue 68 from the patient's body (e.g., the patient's lung) into the tube. Alternatively, tissue may be drawn into the barrel 14 from a patient's body.

Referring now to FIGS. 4-6, when the recesses 50 in the splines 42, 44, 46 and 48 become axially aligned with the slots 29 in the flange 26, the axial movement of the plunger 16 may be discontinued. The plunger 16 may then be rotated in either a clockwise or a counterclockwise direction so that the straight edges 50a of the recesses 50 dig into the tabs 28 located on the flange 26 to cause retention of the plunger 16 and barrel 14 in a fixed, locked position relative to each other. The rotation of the plunger 16 occurs from the forward unlocked position shown in FIG. 3 to the withdrawn and locked position shown in FIG. 4. Referring now to FIGS. 4, 5 and 6, each tab 28 is provided with a centrally-located detent indicated at 28a. Each detent 28a is of sufficient width and depth to enable the straight edge 50a of the recess 50 to be engaged therein after rotation to arrest further rotational movement of the splines 42, 44, 46 and 48 and to prevent inadvertent movement of the splines out of the locked position within the tab 28. In this manner, the plunger 16 is prevented from being pulled back into the barrel 14. Thus, a self-locking feature is provided. As the recesses 50 are positioned at predetermined locations over the length of each spline 42, 44; 46 and 48, the plunger 16 may be locked at any one of several locations over the length of the plunger 16. Thus, a medical procedure can proceed without the doctor having to worry about the plunger 16 moving spontaneously forward to reduce the suction pressure. When it is desired to move the plunger 16 forward again, one only has to rotate it so that the splines 42, 44, 46 and 48 are brought out of their gripping relation with the tabs 28. Then the plunger 16 may be readily advanced again, or further retracted, as desired. With the plunger 16 in the position shown in FIG. 4, any fluid 68 or tissue in the barrel 14 is retained for subsequent testing.

The self-locking syringe assembly 10 described above has certain important advantages. It can be pre-sterilized and pre-packaged. It can be brought into the operating room and the package can be opened under sanitary conditions. It can be used relatively easily and efficiently to collect a sample of fluid and tissue from the patient's body and then it can be further operated to retain the fluid or tissue in the barrel 14.

When the self-locking syringe assembly 10 has been used and the fluid or tissue has been removed from the barrel 14, the assembly 14 can be discarded. The syringe assembly 10 of the present invention can be made economically and relatively compact so that it can be easily stored. It is also light in weight. This facilitates the ease with which the assembly 10 can be operated to withdraw the fluid or tissue into the barrel 14.

Although this invention has been disclosed and illustrated with reference to particular embodiments, the principals involved are susceptible for use in numerous other embodiments which will be apparent to persons skilled in the art. The invention is, therefore, to be limited only as indicated by the scope of the appended claims. The described embodiment is to be considered in all respects only as illustrative and not restrictive. The scope of the invention is indicated by the appended claims rather than by the foregoing description.

I claim:

1. A syringe assembly comprising:
a. a barrel having an open proximal al end, a distal end, and a sidewall extending therebetween, said sidewall having an interior surface defining a hollow internal chamber;
b. an elongate plunger shaft having a distal tip and a proximal end extending beyond the barrel open proximal end, said plunger shaft being axially moveable in both proximal and distal directions within said barrel, and having at least two longitudinally extending splines;
c. a stopper mounted to said distal tip and configured to slidably engage the sidewall interior surface and forming a fluid-tight seal therewith;
d. a flange located at the proximal end of said barrel, said flange having a central opening;
e. at least two tabs positioned at annularly spaced location on said flange central opening, said tabs extending radially inwardly therefrom a predetermined distance and defining slots therebetween, said slots provided for receiving said splines during axial movement of said plunger shaft within said barrel; and,
f. said splines including recesses to provide for rotational movement of said plunger shaft when said recesses are axially aligned with said tabs to dispose said splines against said tabs whereby said plunger shaft is locked in a predetermined. longitudinal position relative to said barrel and prevented from being pulled or pushed towards the distal end of said barrel.

2. The syringe assembly of claim 1, wherein the sidewall outer surface includes graduation marks correlating to an amount of liquid or tissue material withdrawn.

3. The syringe assembly of claim 1, wherein said plunger shaft includes a disc shaped thumb support located at its proximal end, said thumb support configured to limit distal movement of the plunger shaft.

4. The syringe assembly of claim 1, wherein said elongated plunger additionally comprises a stopper mount at its distal tip thereof, said stopper mount comprising at least a spoke structure formed of radially extending spokes for supporting said stopper.

5. The syringe assembly of claim 4, wherein said radially extending spokes includes four radially extending spokes forming a cross pattern for mounting said stopper thereon.

6. The syringe assembly of claim 1, wherein said stopper head is formed of an elastomeric material.

7. The syringe assembly of claim 1, wherein said at least two longitudinally extending splines includes three splines forming a “Y” pattern.

8. The syringe assembly of claim 1, wherein said at least two longitudinally extending splines includes four splines forming an “X” pattern.

9. The syringe assembly of claim 8, wherein each said tab additionally comprises a detent therein, said detent arranged to arrest rotational movement of said spline.

10. The syringe assembly of claim 1, wherein each said recess of each said spline includes a straight edge and an inclined edge.
